(19) United States
(12) Patent Application Publication
(10) Pub. No.: US 2024/0390645 A1
(43) Pub. Date: Nov. 28, 2024

(54) Instrument Advancement Device with Light-Enabled Instrument Viewing

(71) Applicant: Becton, Dickinson and Company, Franklin Lakes, NJ (US)

(72) Inventors: Austin Paul Newton, Layton, UT (US); Curtis H. Blanchard, Herriman, UT (US); Adam J. Boud, Bluffdale, UT (US)

(21) Appl. No.: 18/657,274

(22) Filed: May 7, 2024

Related U.S. Application Data
(60) Provisional application No. 63/468,097, filed on May 22, 2023.

(51) Int. Cl. A61M 25/01; A61M 39/02

(52) U.S. Cl. CPC A61M 25/0105; A61M 39/0247; A61M 2025/0166; A61M 2039/0258; A61M 2205/587

(57) ABSTRACT

Provided herein is an instrument delivery device for use with an intravenous catheter assembly. The instrument delivery device includes a housing having a proximal end portion and a distal end portion and defining an inner volume, an instrument movably received within the inner volume, a coupling device positioned at the distal end portion of the housing and configured to couple the housing to an access connector of an intravenous catheter assembly, an advancement member configured to move relative to the housing to advance a distal end of the instrument beyond the distal end portion of the housing and into the intravenous catheter assembly, and a light generating element disposed at least partially within the housing and configured to generate light that enables visualization of the instrument.

### CROSS-REFERENCE TO RELATED APPLICATION

The present application claims priority to U.S. Provisional Application No. 63/468,097, entitled “Instrument Advancement Device with Light-Enabled Instrument Viewing”, filed May 22, 2023, the entire disclosure of which is hereby incorporated by reference.

### BACKGROUND OF THE INVENTION

### Field of the Invention

Provided herein are instrument advancement devices for use in vascular access, and, in particular, instrument advancement devices incorporating components for light-enabled instrument viewing.

### Description of Related Art

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

As known in the art, instrument advancement devices are often used in connection with an in-dwelling PIVC to facilitate blood draw and/or administering of fluids. When such instrument advancement devices are used to facilitate blood draw, the devices (i.e., “blood draw devices) have focused on the ability to reliably collect a high-quality blood sample and reduce hemolysis. The main method by which these instrument advancement devices work is by employing an introducer for inserting a catheter, probe, tube, or other instrument through the catheter lumen of the PIVC, with the introducer being attached to a catheter adapter of the PIVC that provide for insertion of the catheter. For example, the catheter adapter may include a needleless access connector thereon by which the catheter may be introduced to provide access to the PIVC and into the patient's vasculature. If performing a blood draw, a syringe or vacutainer may then be used to collect blood samples without needing to subject the patient to additional needle sticks.

The introducer of an instrument advancement device typically includes a housing, an instrument (i.e., a catheter or other probe, tube, or instrument) movable within the housing so as to be extendable out therefrom for advancement into the in-dwelling PIVC, and an advancement member that may be actuated by an operator relative to the housing. That is, the advancement member may be moved distally by the operator to cause a corresponding movement of the instrument relative to the housing, such that the instrument may be advanced out from the housing and into the in-dwelling PIVC.

It is recognized that instrument advancement devices may be employed in many different environments and in varying conditions. For example, blood draws may be required at night or in surroundings where there is a lack of light, thereby resulting in reduced/poor visibility for a clinician operating the introducer. Under such conditions, it may be difficult for the clinician to visualize the catheter (or other instrument) as it is advanced through the housing, such that the clinician may not be able to determine the position of the distal tip of the catheter and its position relative to the instrument advancement device and in-dwelling PIVC. Additionally, if during insertion, the tip of the instrument hits an obstruction that requires remediation or positional adjustments to successfully advance, this need for remediation/adjustments may not be obvious without visualization.

Accordingly, a need exists in the art for an instrument advancement device and introducer thereof that provides for improved visibility during operation of the device, including being able to visualize the distal end (or other portions) of a catheter or other instrument as it is advanced by operation of the introducer.

### SUMMARY OF THE INVENTION

Provided herein is an instrument delivery device for use with an intravenous catheter assembly. The instrument delivery device includes a housing having a proximal end portion and a distal end portion and defining an inner volume, an instrument movably received within the inner volume, a coupling device positioned at the distal end portion of the housing and configured to couple the housing to an access connector of an intravenous catheter assembly, an advancement member configured to move relative to the housing to advance a distal end of the instrument beyond the distal end portion of the housing and into the intravenous catheter assembly, and a light generating element disposed at least partially within the housing and configured to generate light that enables visualization of the instrument.

In some embodiments, the light generating element comprises a lighting element positioned adjacent the distal end portion or the proximal end portion of the housing.

In some embodiments, the instrument delivery device includes an optical fiber extending out from the lighting element and configured to transmit light along a length of the optical fiber.

In some embodiments, the light generating element comprises one or more light emitting diodes (LEDs).

In some embodiments, the one or more LEDS are affixed to an inner surface of the housing.

In some embodiments, the light generating element comprises a light strip a plurality of LEDs arranged on a substrate, the light strip affixed to the inner surface of the housing.

In some embodiments, the advancement member comprises a first portion configured to move along an outer surface of the housing and a second portion connected to the first portion and positioned within the inner volume, the second portion engaging the instrument, wherein the one or more LEDS are positioned on a distal end of the second portion of the advancement member, such that the one or more LEDS move with the advancement member when the advancement member is moved relative to the housing.

In some embodiments, the instrument delivery device includes a battery housed on the second portion of the advancement member to power the LED.

In some embodiments, the one or more LEDS comprise micro-LEDs having dimensions of 1.5 mm×1.5 mm or less.

In some embodiments, the housing is formed of a fluorescent plastic.

In some embodiments, the instrument delivery device includes a power circuit having an open state and a closed state, the power circuit providing power to the light generating element when in the closed state, wherein the power circuit is in the open state when the advancement member is in a home position, at the proximal end portion of the housing, and wherein the power circuit is transitioned from the open state to the closed state when the advancement member is moved distally from the home position, such that the light generating element receives power from the power circuit and is caused to emit light.

In some embodiments, the power circuit is configured to change a light color output from the light generating element based on a positioning of the advancement member.

In some embodiments, the light generating element comprises a luminescent coating applied on at least a portion of the instrument, the luminescent coating generating light after exposure to ambient light.

Also provided herein is a catheter system including an intravenous catheter assembly comprising a catheter comprising a distal end and a proximal end, the catheter defining a lumen extending between the distal end and the proximal end and an access connector configured to provide access to the lumen of the catheter. The catheter system also includes an instrument delivery device coupleable to the access connector, the instrument delivery device comprising a housing having a proximal end portion and a distal end portion and defining an inner volume, an instrument movably received within the inner volume, a coupling device positioned at the distal end portion of the housing and configured to couple the housing to an access connector of an intravenous catheter assembly, an advancement member configured to move relative to the housing to advance a distal end of the instrument beyond the distal end portion of the housing and into the intravenous catheter assembly, and a light generating element disposed at least partially within the housing and configured to generate light that enables visualization of the instrument.

In some embodiments, the light generating element comprises a lighting element positioned adjacent the proximal end portion of the housing and an optical fiber extending out from the lighting element and configured to transmit light along a length of the optical fiber.

In some embodiments, the light generating element comprises one or more light emitting diodes (LEDs), with the one or more LEDS affixed to an inner surface of the housing or to a portion of the advancement member positioned within the inner volume of the housing.

In some embodiments, the one or more LEDS comprise micro-LEDs having dimensions of 1.5 mm×1.5 mm or less, and wherein the instrument advancement device further comprises a battery to power the micro-LEDs.

In some embodiments, the instrument delivery device includes a power circuit having an open state and a closed state, the power circuit providing power to the light generating element when in the closed state, wherein the power circuit is in the open state when the advancement member is in a home position, at the proximal end portion of the housing, and wherein the power circuit is transitioned from the open state to the closed state when the advancement member is moved distally from the home position, such that the light generating element receives power from the power circuit and is caused to emit light.

In some embodiments, the light generating element comprises a luminescent coating applied on at least a portion of the instrument, the luminescent coating generating light after exposure to ambient light.

In some embodiments, the housing is formed of a fluorescent plastic.

### BRIEF DESCRIPTION OF THE DRAWINGS

FIG. 1 is a perspective view of a catheter system, in accordance with an aspect of the disclosure;

FIG. 2 is a perspective view of a blood draw device useable with the catheter system of FIG. 1, in accordance with an aspect of the disclosure;

FIG. 3 is an exploded view of the blood draw device of FIG. 2, in accordance with an aspect of the disclosure;

FIG. 4 is a side view of the blood draw device of FIG. 2, showing the catheter tube in a first, retracted position;

FIG. 5 is a side view of the blood draw device of FIG. 2, showing the catheter tube in a second, extended position;

FIGS. 6A and 6B are perspective views of portions of the blood draw device housing, including lighting elements provided thereon, in accordance with an aspect of the disclosure:

FIGS. 7A and 7B are perspective views of portions of the blood draw device housing, including lighting elements provided thereon, in accordance with an aspect of the disclosure;

FIGS. 8A and 8B are perspective views of portions of the blood draw device housing, including lighting elements provided thereon, in accordance with an aspect of the disclosure;

FIG. 9 is an exploded view of a blood draw device, in accordance with an aspect of the disclosure;

FIG. 10 is a perspective view of the advancement member of the blood draw device of FIG. 9;

FIG. 11 is an exploded view of a blood draw device, in accordance with an aspect of the disclosure;

FIG. 12 is a cross-sectional view of the catheter tube of the blood draw device of FIG. 11, taken along line 12-12, with a luminescent coating on the catheter tube; and

FIG. 13 is a perspective view of a catheter system, in accordance with another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

Provided herein are devices and systems for introducing instruments through indwelling catheters, such as peripheral intravenous catheters (PIVCs), peripherally inserted central catheters (PICCs), central venous catheters (CVCs), and midline catheters. While certain catheter assemblies are shown in the accompanying figures and described below, those of skill will appreciate that a disinfecting probe as described herein may be useful in any number of different catheter assembly configurations.

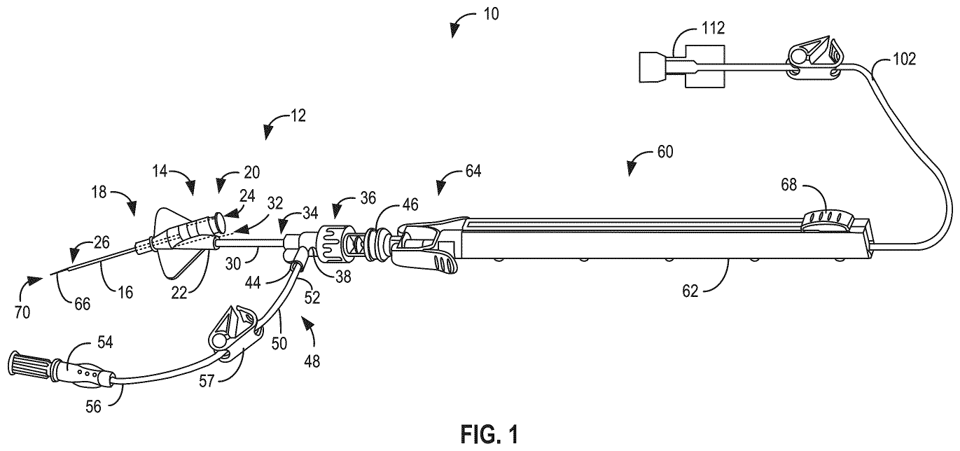

Referring now to FIG. 1, shown is a non-limiting embodiment of a catheter system 10 for facilitating delivery of an instrument into an in-dwelling catheter, such as may be desirable for blood collection (or for other sensing purposes) from the patient. Catheter system 10 may include a catheter assembly 12 having a catheter adapter 14 and associated catheter 16. The catheter adapter 14 may include a distal end 18 and a proximal end 20. In some embodiments, the catheter adapter 14 may include an additional adapter port 22 that may be disposed between the distal end 18 and the proximal end 20 or disposed at the proximal end 20. The catheter adapter 14 may include a first lumen 24 extending through the distal end 18 and the proximal end 20, and the first lumen 24 may be sealed at proximal end 20 of catheter adapter 14. The catheter 16 may be formed of any suitable material and may be of any useful length, as known to those of skill in the art. Catheter 16 extends from the distal end 18 of catheter adapter 14 and may be placed into the vasculature of the patient, with a distal end or tip 26 of the catheter 16 positioned appropriately within a vein to enable a blood draw from the patient.

In some non-limiting embodiments or aspects, the catheter assembly 12 may include a first fluid conduit 30 extending from the port 22. First fluid conduit 30 may be formed of any suitable material known to those of skill in the art and may have a distal end 32 and a proximal end 34. The distal end 32 of first fluid conduit 30 is coupled to port 22, while the proximal end 34 of first fluid conduit 30 may be coupled to a connector 36. Connector 36 may be a t-connector (e.g., one side port arranged at a 90 degree angle relative to a longitudinal axis of connector 36), a y-connector (e.g., one side port arranged at a 25, a 60, or a 75 degree angle relative to a longitudinal axis of connector 36), or any other type of connector known in the art. The connector 36 may include a second lumen 38 therethrough, having any number of branches suitable for the type of connector, such as a branch extending between distal and proximal ends of connector 36 and a branch provided to a port 44 of the connector 36.

In some non-limiting embodiments or aspects, catheter assembly 12 may include a needleless access connector 46 coupled to the proximal end 42 of connector 36, with the needleless access connector 46 providing an access port to the catheter assembly 12. The needleless access connector 46 may be configured as a split-septum connector or self-healing septum connector, as examples. In the illustrated embodiment, the access port provided by needleless access connector 46 is a near-patient access port close to the insertion site of the catheter 16, but it is recognized that an access port could be provided at other alternative locations close enough to the insertion site that allow for advancement of a blood draw catheter tube or other probe into the indwelling catheter 16 and out beyond the distal tip thereof. For example, an access port that provides for advancement of a blood draw catheter tube into the indwelling arterial catheter 16 could be located on another connector, such as a proximal connector on an extension set (as described below) of the catheter assembly 12.

In some non-limiting embodiments or aspects, catheter assembly 12 may also include an extension set 48 coupled to the port 44 of the connector 36. The extension set 48 may include a second fluid conduit 50 coupled to port 44 at end 52 of the conduit 50 and a luer connection 54 at opposing end 56, with a clamp 57 provided on second fluid conduit 50 that allows for occlusion thereof.

Catheter system 10 further includes an instrument delivery device 60 that may be operated to introduce an instrument into the catheter assembly 12 and into the vasculature of the patient. In some embodiments, and as referred to hereafter, the instrument delivery device 60 may comprise a blood draw device (“blood draw device 60”) that introduces a catheter tube through the catheter assembly 12 and into the patient vasculature to obtain a blood sample from the patient. However, it is recognized that the instrument delivery device 60 could instead introduce a guidewire, probe, or other sensor into the patient vasculature, according to aspects of the disclosure, and thus it is to be understood that the below description of a blood draw device should not limit the scope of the disclosure.

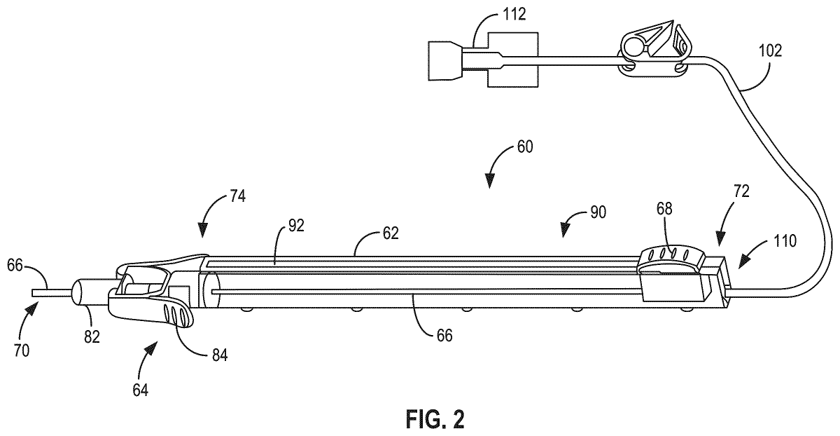

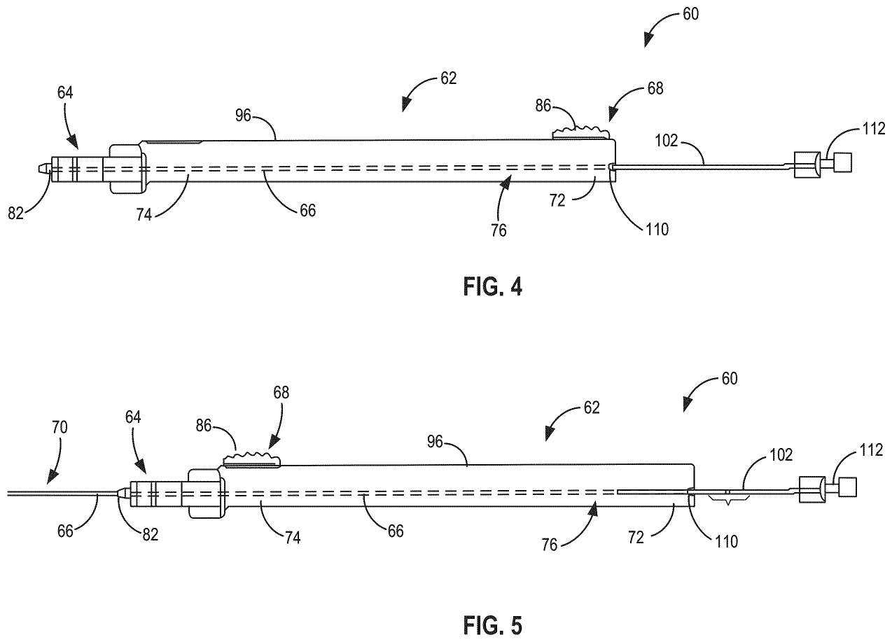

As shown in FIG. 1 and in further detail in FIGS. 2-4, according to a non-limiting embodiment, the blood draw device 60 includes at least a housing 62, a coupling device 64, a catheter tube 66, and an advancement member 68. As will be described in further detail below, the catheter tube 66 is moveable within the housing 62 so as to provide for advancement of a portion of the catheter tube 66 from a first or retracted position inside the housing 62 (FIG. 4 to a second or advanced position outside of the housing 62 (FIG. 5), such that a distal end thereof may be routed into the catheter assembly 12. Once a portion of the catheter tube 66 has been routed into the catheter assembly 12 and out past the distal tip 26 of indwelling catheter 16, the catheter tube 66 may enable collection of a blood sample.

According to embodiments, the catheter tube 66 is sized to enable introduction thereof into the fluid path (i.e., into a lumen of catheter 16, lumen 24 of catheter adapter 14, and first fluid conduit 30) of catheter assembly 12 and for advancement therethrough. Accordingly, the catheter tube 66 can have an outer diameter (e.g., between a 10-gauge and a 30-gauge) that is smaller than the smallest lumen of the catheter assembly fluid path. The catheter tube 66 can have a length that is sufficient to place a distal end 70 of the catheter tube 66 in a desired position within the fluid path of the catheter system 10. Thus, in one embodiment, the catheter tube 66 may have a length sufficient to provide for advancement of the distal end 70 thereof out from the housing 62 and through the catheter assembly (i.e., through connector 36, fluid conduit 30, catheter adapter 14 and catheter 16), and all the way out past the distal tip 26 of catheter 16.

As shown in FIGS. 1-5, the housing 62 of blood draw device 60 can be an elongate member having a proximal end 72 and a distal end 74 and defining an inner volume 76. In some embodiments, the housing 62 may be formed of a pair of housing portions 78a, 78b that are coupled together to define the inner volume 76. The housing 62 may include one or more features or surface finishes on an outer surface thereof that can be arranged to increase the ergonomics of the blood draw device 60, which in some instances can allow a user to manipulate the blood draw device 60 with one hand (i.e., single-handed use). Additionally, the housing 62 may be constructed of a transparent or translucent material (e.g., fluorescent plastic) that provides for at least partial visibility into the inner volume 76 thereof, such that positioning of catheter tube 66 within the housing 62 can be ascertained by a clinician, as explained in further detail below.

The coupling device 64 of blood draw device 60 is provided at the distal end 74 of the housing 62, with the coupling device 64 providing for reversible coupling of the blood draw device 60 to catheter assembly 12, such as via needleless access connector 46 as shown in FIG. 1. In some embodiments, the coupling device 64 is configured as a lock 80 that includes a blunted cannula 82 and locking arms 84 for coupling to the needleless access connector 46 of catheter assembly 12, with the blunted cannula 82 and locking arms 84 forming three points of contact therewith. However, those of skill will appreciate that any connection or coupling, for example a luer, can be used, so long as the distal end 70 of catheter tube 66 may pass through the coupling device 64 to catheter assembly 12.

The advancement member 68 of blood draw device 60 includes a first portion 86 and a second portion 88. The first portion 86 is movably disposed along an upper surface 90 of the housing 62 and the second portion 88 is movably disposed within the inner volume 76 of the housing 62. The arrangement of the advancement member 68 and the housing 62 is such that a connecting portion (not shown) of the advancement member 68 that joins the first and second portions 86, 88 is seated within a slot 92 formed in the upper surface 90 of the housing 62—the slot 92 generally extending between the proximal and distal ends 72, 74 of the housing 62. As the first and second portions 86, 88 are joined together, movement of the first portion 86 along the upper surface 90 of the housing 62 results in a corresponding movement of the second portion 88 within the inner volume 76.

As shown in FIGS. 1-5, the first portion 86 of the advancement member 68 may be configured as a tab having a contact surface 94a engageable by a user and an underside 94b that is in contact with the outer surface 90 of the housing 62. In such embodiments, the upper surface 90 of the housing 62 can include a track 96, for example, a set of ribs, ridges, bumps, grooves, and/or the like along which the underside 94b of tab or protrusion advances when the advancement member 68 is engaged by a user. In this manner, a user can engage the first portion 86 of the advancement member 68 and can move the advancement member 68 relative to the housing 62.

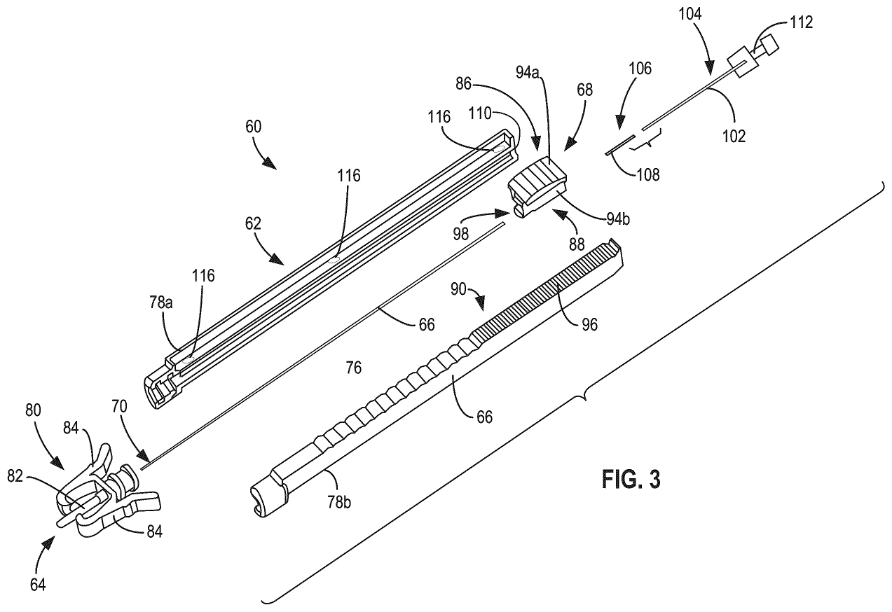

As shown in FIG. 3, the second portion 88 includes an opening 98 extending therethrough that is configured to grip or retain a portion of the catheter tube 66. Due to a portion of the catheter tube 66 being retained within the opening 98 of second portion, 94, movement of the advancement member 68 relative to housing 62 causes a corresponding movement of the catheter tube 66 relative to the housing 62. In this manner, the distal end 70 of the catheter tube 66 can be selectively moved out of or back into the inner volume 76 of the housing 62 as desired, such as advancing the distal end 70 of the catheter tube 66 out of the housing 62 when the blood draw device 60 has been coupled to the catheter assembly 12 and collection of a blood sample is to be performed.

As further shown in FIGS. 1-5, blood collection device 60 includes a secondary catheter 102 provided at the proximal end 72 of the housing 62. The secondary catheter 102 has a proximal end portion 104 and a distal end portion 106 and defines a lumen 108. A portion of the secondary catheter 102 is disposed within and extends through an opening 110 formed in the proximal end 72 of housing 62. As such, the proximal end portion 104 is at least partially disposed outside of the housing 62 and the distal end portion 106 is at least partially disposed within the housing 62, with the distal end portion 106 coupled to the second portion 88 of the advancement member 68. In some embodiments, the secondary catheter 102 can have a larger diameter than the catheter tube 66, which can function to limit, reduce, and/or substantially prevent hemolysis of a volume of blood as the volume of blood flows through the catheter 66 and the secondary catheter 102. According to embodiments of the disclosure, the proximal end portion 104 of the secondary catheter 102 is coupled to and/or otherwise includes a coupler 112 configured to mate with a collection device (not shown) that is useable with the blood draw device 60 to collect a blood sample for subsequent analysis. According to some embodiments, the coupler 112 may be configured as a luer connection (i.e., a female luer connection) configured to mate with a corresponding luer connection (i.e., a male luer connection) of the collection device.

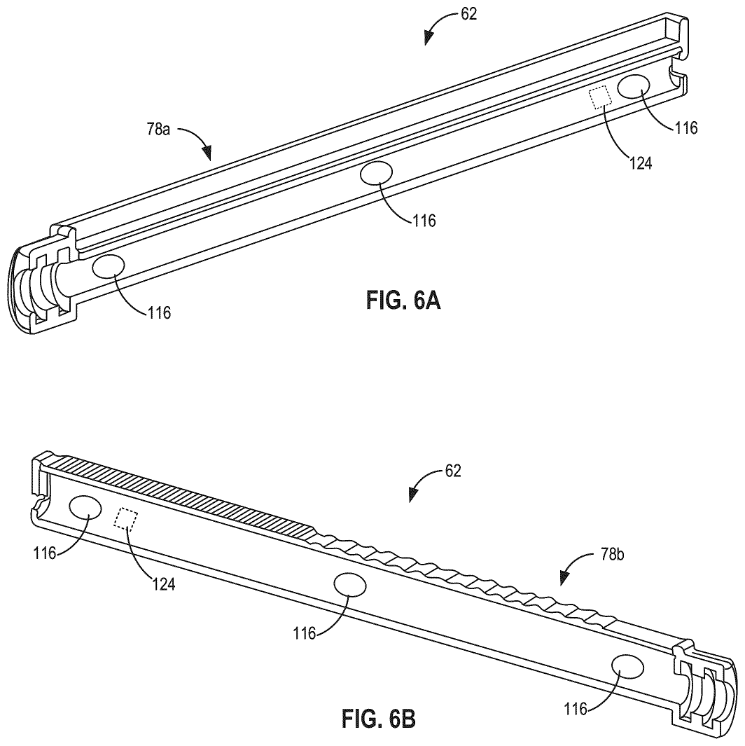

According to aspects of the disclosure, and referring now to FIGS. 6-12, blood draw device 60 is configured to incorporate components or elements therein that provide for light-enabled viewing of the catheter tube 66 during operation of the device 60—i.e., “light generating elements.” That is, light generating elements are provided in blood draw device 60 that are configured to generate light/illumination sufficient to view the catheter tube 66 during operation of the blood draw device 60, such that positioning of the catheter tube 66 can be determined as the catheter tube 66 is advanced and/or retracted relative to the housing 62 and into catheter assembly 12.

In accordance with one embodiment, blood collection device 60 includes one or more lighting elements 116 therein positioned within housing 62 that are configured to emit light therefrom. According to embodiments of the disclosure, lighting element(s) 116 may be configured as any of a number of suitable light sources; however, in an exemplary embodiment, lighting element(s) 116 may be provided as light emitting diodes (LEDs) (hereafter “LEDs 116”).

According to some embodiments, the LED(s) 116 may be secured to housing 62 within the inner volume 76, such as being affixed to the inner surface of the housing 62 at one or more locations thereof. As shown in phantom in FIG. 3 and FIGS. 6A and 6B, one or more LEDs 116 may be provided at the proximal end 72 of housing 62, the distal end 74 of housing 62, and/or at locations of housing 62 between the proximal and distal ends.

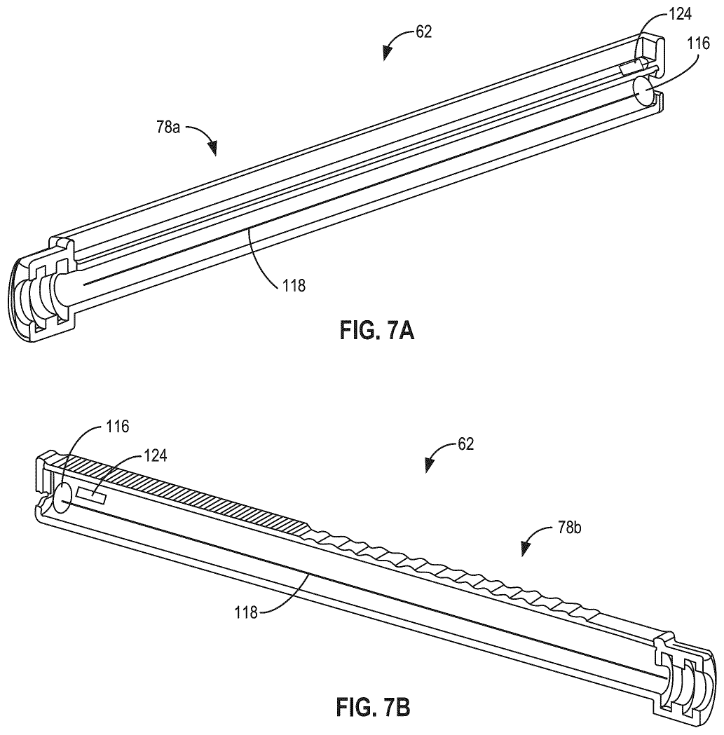

As shown in FIGS. 7A and 7B, in accordance with one aspect of the disclosure, when an LED 116 (or LEDs 116) is positioned at one end of the housing 62, such as at proximal end 72, an optical fiber 118 may be provided (for each lighting element 116) that is connected to the LED 116. The optical fiber 118 is composed of one or more glass fibers of a composition and thickness that provide for light transmission (from lighting element 116) along a length thereof. In some embodiments, the optical fiber 118 may be configured such that light is emitted out from the optical fiber 118 along an entire length thereof. In other embodiments, portions of the optical fiber 118 may be surrounded by a cladding layer (not shown) composed of one or more layers of materials having a lower refractive index than that of the glass fibers of optical fiber 118, with cladding provided at locations spaced apart along the length of the optical fiber 118, such that light is emitted out from the optical fiber 118 at the locations of openings in the cladding.

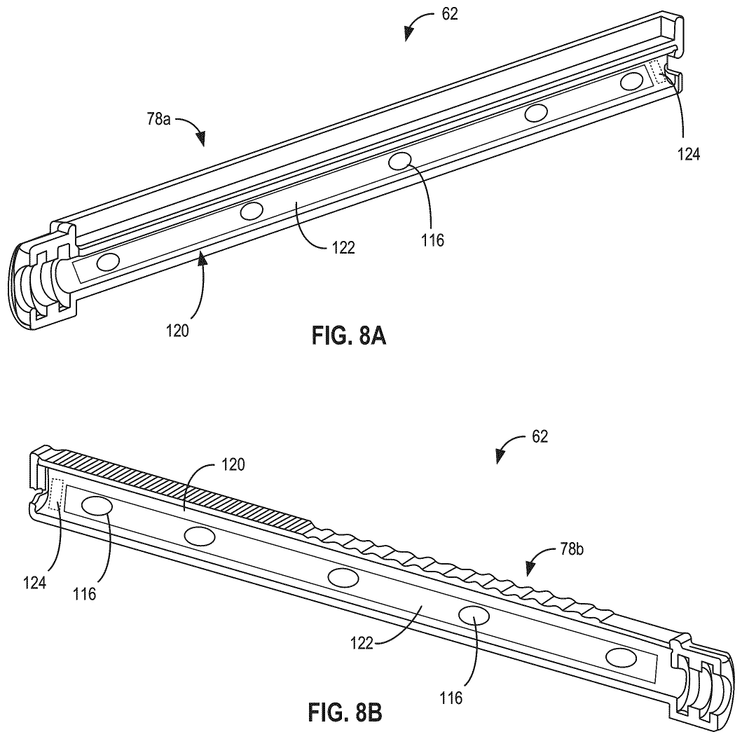

As shown in FIGS. 8A and 8B, in some embodiments, a plurality of LEDs 116 may be provided as part of a light strip 120 that is secured to an interior surface of housing 62. The light strip 120 may comprise a substrate 122 on which the plurality of LEDs 116 are positioned. In some embodiments, the LEDs 116 may be equally spaced along a length of the substrate 122, with the light strip 120 extending longitudinally along housing 62, at least partially between the proximal and distal ends 72, 74 thereof. In other embodiments, the LEDs 116 may be positioned on substrate 122 so that the light strip 120 illuminates certain critical advancement points or regions where the catheter tube 66 is most likely to experience bowing and bending.

According to embodiments of the disclosure, the LEDs 116 may be configured as micro-LEDs sized to fit within housing 62, due to the smaller size of the blood draw device 60 (and housing 62 thereof). The micro-LEDs may have dimensions of 1.5 mm×1.5 mm (0.06 in ×0.06 in), for example, so as to allow the LEDs 116 to be incorporated into the existing architecture of the blood draw device 60.

According to aspects of the disclosure, the LEDs 116 may be powered by an accompanying power system that, in an exemplary embodiment, may be provided as one or more batteries 124 that provide power to the LED(s) 116. The battery or batteries 124 may be configured as coin cell batteries or watch batteries, as non-limiting examples, having a reduced size (e.g., 6.8 mm (0.27 in) diameter), so as to again allow for positioning thereof within housing 62.

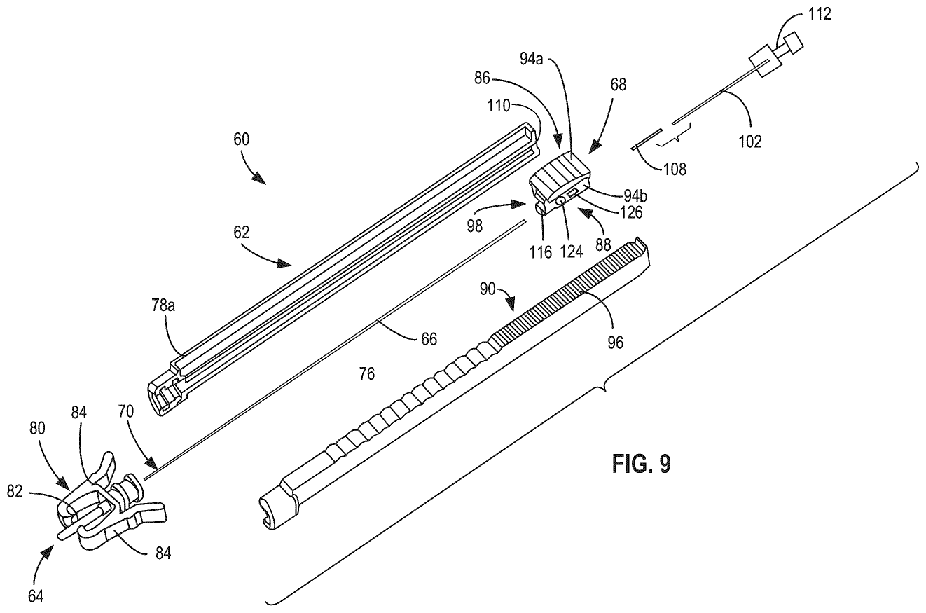

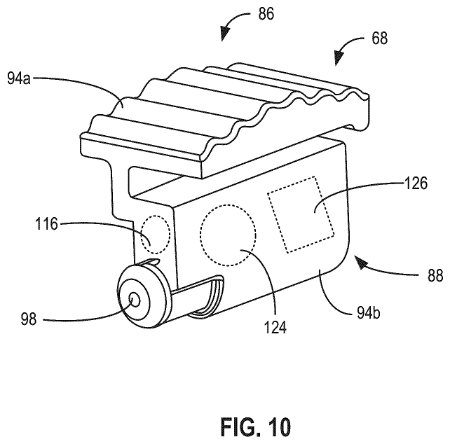

As shown in FIGS. 9 and 10, in some embodiments, one or more LEDs 116 may be secured to advancement member 68 (i.e., to second portion 88 thereof), such that LED(s) 116 would move along with advancement member 68 when the advancement member is translated relative to housing 62. The LED(s) 116 may be positioned on a distal-facing surface of the second portion 88 of advancement member 68—within the inner volume 76 of housing 62. In some embodiments, battery 124 may also be provided on or as part of the advancement member 68. The battery 124 may be accommodated in a recess formed in the second portion 88, such as on a side-facing surface of the second portion 88 (FIG. 10), so as to not interfere with movement of the advancement member 68 relative to housing 62.

In accordance with some aspects of the disclosure, and as shown in FIGS. 9 and 10, a power circuit 126 is provided in instrument delivery device 60 that selectively provides power from a power source (e.g., battery 124) to the LED(s) 116 during operation of the device 60. Power circuit 126 may be configurable between an open state and a closed state, such as via one or more switches (not shown) included therein. The power circuit 126 may thus operate to provide/transfer power to the LED(s) 116 when in the closed state and prevent power from being provided/transferred to the LED(s) 116 when in the open state.

In some embodiments, operation/configuring of the power circuit 126 in the open and closed states, and switching of the power circuit 126 from the open state to the closed state, may be controlled based on positioning of the advancement member 68 relative to housing 62. In one embodiment, the power circuit 126 may be configured in the open state when the advancement member 68 is in a home position, at the proximal end portion 72 of the housing 62, and the power circuit 126 may be transitioned from the open state to the closed state responsive to the advancement member 68 being moved distally from the home position, i.e., advanced toward the distal end portion 74 of housing 62, such that the LED(s) 116 receives power from the power circuit 126 and is/are caused to emit light. Thus, in an embodiment where LED(s) 116 are positioned on the advancement member 68, the LED(s) 116 may be powered on responsive to distal movement of advancement member 68 (to advance catheter tube 66)—with the power circuit 126 being configured in the closed state when the advancement member 68 is moved distally from its home position and thereby transferring power to the LED(s) 116. In other embodiments, the LED(s) 116 may be powered on when advancement of the catheter tube 66 is adequate to consider beginning a blood draw. In such an embodiment, power circuit 126 may be transitioned from the open state to the closed state when the advancement is deemed adequate, when a feature of the catheter tube 66 is exposed to the blood of the patient, or from some other signal in the catheter. In some embodiments, in addition to powering on of the LED(s) 116 by power circuit 126, the power circuit 126 (or another associated control circuit) could control an illumination of the LED(s) 116, such as a color light output therefrom. For example, the LED(s) could alternate colors, with the LED(s) 116 illuminating in red for an initial advancement of the catheter tube 66 and illuminating in green when the catheter tube 66 has advanced adequately to perform a blood draw (or take a reading from a sensor thereon, when the instrument is a probe). In still other embodiments, the power circuit 126 could cause an indicator light to illuminate when the catheter tube 66 has advanced adequately to perform a blood draw.

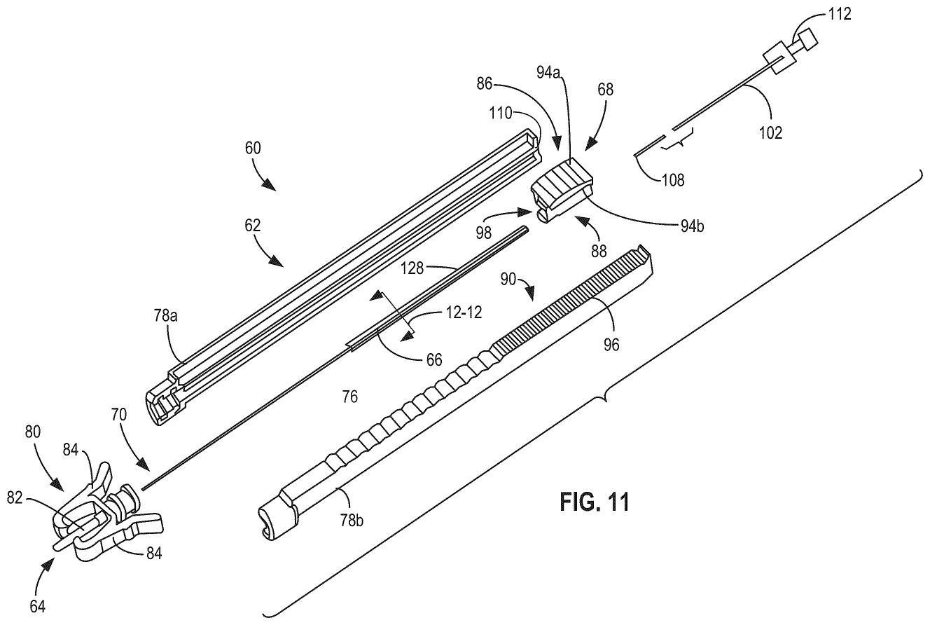

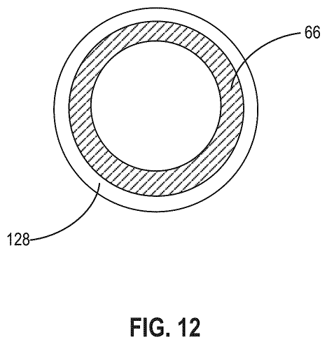

Referring now to FIGS. 11 and 12, in accordance with an aspect of the disclosure, a light generating element is provided in blood draw device 60 in the form of a luminescent material or coating 128 that applied is applied to one or more components in the blood draw device 60. The luminescent material 128 is configured to emit light therefrom responsive to exposure to an ambient light. According to non-limiting examples, the bio-compatible luminescent material 128 may be any of a number of suitable long-persistent luminescent (LPL) materials, including tetramethylbenzidine (TMB), bis(diphenylphosphoryl)dibenzo[b,d] thiophene (PPT), and/or a mixture of TMB/PPT. In some embodiments, the luminescent material 128 is applied to at least a portion of the catheter tube 66 and, in such embodiments, the luminescent material 128 is provided as a bio-compatible luminescent material. According to embodiments, an entirety of the catheter tube 66 may be coated with the bio-compatible luminescent material 128, or only a desired section of the catheter tube 66 may be coated with the bio-compatible luminescent material 128, such as the proximal end/section of the catheter tube 66 (that is visible in the housing during advancement/insertion) being coated with the bio-compatible luminescent material 128, while the distal end/section of the catheter tube 66 that enters the patient vasculature is not treated. In other embodiments, the luminescent material 128 may be applied to the housing 62 at desired locations, so as to provide for illumination of the catheter tube 66 during operation of the blood draw device 60.

In accordance with some aspects of the disclosure, in order to aid in visualization of the catheter tube 66 during operation of the instrument delivery device 60, the housing 62 may be formed as a fluorescent plastic housing. The fluorescent plastic housing 62 may be illuminated by lighting elements 116 provided in the inner volume 76 of the housing and may further allow for visualization of the catheter tube 66 through the housing 62.

Therefore, according to embodiments described herein, the blood draw device 60 is configured to provide for light-enabled viewing of the catheter tube 66 as the catheter tube 66 is advanced into and through a catheter assembly and into the vein of a patient. Light generating elements are provided in blood draw device 60 that are configured to generate light/illumination sufficient to view the catheter tube 66 during operation of the blood draw device 60, such that positioning of the catheter tube 66 can be determined as the catheter tube 66 is advanced and/or retracted relative to the housing 62 and into catheter assembly 12. In some embodiments, the catheter tube 66 is illuminated in order to assist in visualizing the catheter tube 66, while in other embodiments a background (e.g., parts of the housing 62) may be illuminated so that the catheter tube 66 appears black/dark against the illuminated background, to assist in visualizing the catheter tube 66.

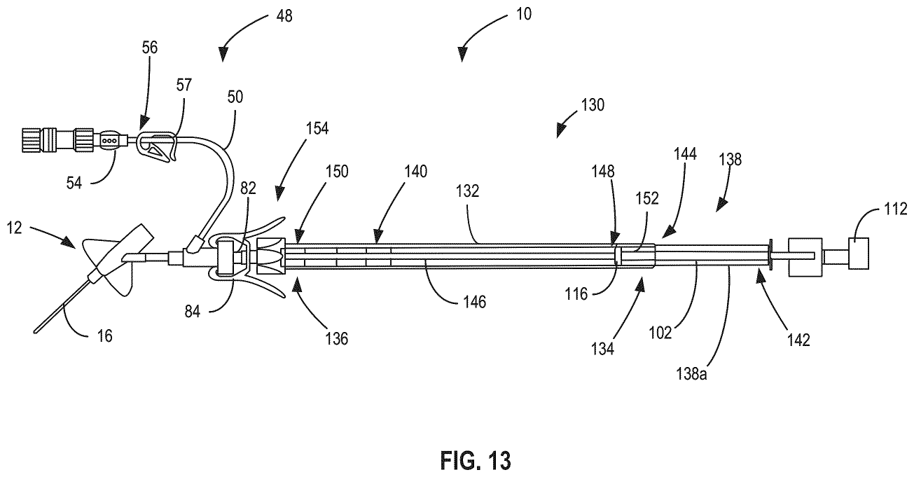

It is recognized that aspects of the disclosure are not limited to the specific blood draw device 60 shown and described in FIGS. 1-12, and that blood draw devices with other suitable constructions may also incorporate aspects of the disclosure. Referring now to FIG. 13, a catheter system 10 is shown that includes a blood draw device 130 according to another embodiment of the disclosure. The blood draw device 130 includes a housing 132 having a proximal end 134 and a distal end 136, and an advancement member 138 slideably received within housing 132 (i.e., within an inner volume 140 of housing 132). In the illustrated embodiment, the advancement member 138 is provided as one or more telescopic cylinders 138a that are provided in a telescoping relationship with housing 132, such that advancement member 138 may be slideably received entirely, or almost entirely, within the inner volume 140 of housing 132. Advancement member 138 also includes a proximal end 142 and a distal end 144 and, in non-limiting embodiments, advancement member 138 may have a variable diameter along its length. As one example, the distal end 144 of advancement member 138 may have a larger diameter than other portions of advancement member 138 such that, as advancement member 138 is retracted, one or more features on housing 132 may interact with the enlarged portion of advancement member 138 to prevent pulling advancement member 138 completely out of housing 132. As another example, the distal end 144 of advancement member 138 may have a smaller diameter than other portions of advancement member 138, to keep the advancement member 138 in position at a blood draw forward condition, so that a hand of the operator is freed up to manipulate additional components (e.g., a vacutainer tube).

Blood draw device 130 further includes a catheter tube 146 having a proximal end 148 and a distal end 150. Catheter tube 146 is received within the inner volume 140 of housing 132, and may be advanced and/or retracted relative to housing 132 by displacement of the advancement member 138 relative to the housing 132. In some embodiments, the catheter tube 146 may be joined to advancement member 138 via a fitting 152 provided at the distal end 144 of advancement member 138, such that displacement of the advancement member 138 relative to the housing 132 causes a corresponding displacement of catheter tube 146. In non-limiting embodiments, catheter tube 146 may be advanced from a first positon in which distal end 150 of catheter tube 146 is within housing 132, to a second position in which a distal end 150 of catheter tube 146 is positioned distally of housing 132 (and also positioned distally of catheter 16), as previously described regarding blood draw device 60 and operation thereof.

Blood draw device also includes a coupling device 154 thereon which may be identical to the coupling device 64 shown and described in the blood draw device of FIGS. 1-12. That is, coupling device 154 is configured as a lock 80 that includes a blunted cannula 82 and locking arms 84 for coupling to the needleless access connector 46 of catheter assembly 12, with the blunted cannula 82 and locking arms 84 forming three points of contact therewith. However, it is appreciated that alternative embodiments of blood draw device 130 may include a coupling device 154 of another type to secure blood draw device 130 to catheter assembly 12, including luer connections, clips, blunt plastic cannulae, blunt metal cannulae, hybrid luers (e.g., with a cannula) friction fits, and the like. According to aspects of the disclosure, a secondary catheter 102 may be routed through the telescopic cylinder 138a of advancement member 138, with the secondary catheter 102 providing a fluid connection between the catheter tube 146 and a coupler 112 provided at the proximal end portion 104 of the secondary catheter 102—with the coupler 112 configured to mate with a collection device (not shown).

As described in detail above, blood draw device 130 may incorporate one or more light generating elements therein that are configured to generate light/illumination sufficient to view the catheter tube 146 during operation of the blood draw device 130, such that positioning of the catheter tube 146 can be determined as the catheter tube 146 is advanced and/or retracted relative to the housing 132 and into catheter assembly 12. According to some embodiments, LEDs 116 may be provided within the inner volume 140 of housing 132 (on an inner surface of housing 132 and/or on a distal-facing surface of advancement member 138, for example) that emit light therefrom to provide for visualization of the catheter tube 146. According to other embodiments, a bio-compatible luminescent material or coating (not shown) may be applied to at least a portion of the catheter tube 146, with the bio-compatible luminescent material emitting light therefrom responsive to exposure thereof to an ambient light.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device for use with an intravenous catheter assembly, the instrument delivery device comprising:
a housing having a proximal end portion and a distal end portion, the housing defining an inner volume;
an instrument movably received within the inner volume;
a coupling device positioned at the distal end portion of the housing and configured to couple the housing to an access connector of an intravenous catheter assembly;
an advancement member configured to move relative to the housing to advance a distal end of the instrument beyond the distal end portion of the housing and into the intravenous catheter assembly; and
a light generating element disposed at least partially within the housing and configured to generate light that enables visualization of the instrument.

2. The instrument delivery device of claim 1, wherein the light generating element comprises a lighting element positioned adjacent the distal end portion or the proximal end portion of the housing.

3. The instrument delivery device of claim 2, further comprising an optical fiber extending out from the light source and configured to transmit light along a length of the optical fiber.

4. The instrument delivery device of claim 1, wherein the light generating element comprises one or more light emitting diodes (LEDs).

5. The instrument delivery device of claim 4, wherein the one or more LEDS are affixed to an inner surface of the housing.

6. The instrument delivery device of claim 4, wherein the light generating element comprises a light strip including a plurality of LEDs arranged on a substrate, the light strip affixed to the inner surface of the housing.

7. The instrument delivery device of claim 4, wherein the advancement member comprises a first portion configured to move along an outer surface of the housing and a second portion connected to the first portion and positioned within the inner volume, the second portion engaging the instrument; and
wherein the one or more LEDS are positioned on a distal end of the second portion of the advancement member, such that the one or more LEDS move with the advancement member when the advancement member is moved relative to the housing.

8. The instrument delivery device of claim 7, further comprising a battery housed on the second portion of the advancement member to power the LED.

9. The instrument delivery device of claim 4, wherein the one or more LEDS comprise micro-LEDs having dimensions of 1.5 mm×1.5 mm or less.

10. The instrument delivery device of claim 4, further comprising a power circuit having an open state and a closed state, the power circuit providing power to the light generating element when in the closed state;
wherein the power circuit is in the open state when the advancement member is in a home position, at the proximal end portion of the housing; and
wherein the power circuit is transitioned from the open state to the closed state when the advancement member is moved distally from the home position, such that the light generating element receives power from the power circuit and is caused to emit light.

11. The instrument delivery device of claim 10, wherein the power circuit is configured to change a light color output from the light generating element based on a positioning of the advancement member.

12. The instrument delivery device of claim 1, wherein the light generating element comprises a luminescent coating applied on at least a portion of the instrument, the luminescent coating generating light after exposure to ambient light.

13. The instrument delivery device of claim 1, wherein the housing is formed of a fluorescent plastic.

14. A catheter system, comprising:
an intravenous catheter assembly comprising:
a catheter comprising a distal end and a proximal end, the catheter defining a lumen extending between the distal end and the proximal end; and
an access connector configured to provide access to the lumen of the catheter; and
an instrument delivery device coupleable to the access connector, the instrument delivery device comprising:
a housing having a proximal end portion and a distal end portion, the housing defining an inner volume;
an instrument movably received within the inner volume;
a coupling device positioned at the distal end portion of the housing and configured to couple the housing to an access connector of an intravenous catheter assembly;
an advancement member configured to move relative to the housing to advance a distal end of the instrument beyond the distal end portion of the housing and into the intravenous catheter assembly; and
a light generating element disposed at least partially within the housing and configured to generate light that enables visualization of the instrument.

15. The catheter system of claim 14, wherein the light generating element comprises:
a lighting element positioned adjacent the proximal end portion of the housing; and
an optical fiber extending out from the lighting element and configured to transmit light along a length of the optical fiber.

16. The catheter system of claim 14, wherein the light generating element comprises one or more light emitting diodes (LEDs), with the one or more LEDS affixed to an inner surface of the housing or to a portion of the advancement member positioned within the inner volume of the housing.

17. The catheter system of claim 16, wherein the one or more LEDS comprise micro-LEDs having dimensions of 1.5 mm×1.5 mm or less, and wherein the instrument advancement device further comprises a battery to power the micro-LEDs.

18. The catheter system of claim 15, further comprising a power circuit having an open state and a closed state, the power circuit providing power to the light generating element when in the closed state;
wherein the power circuit is in the open state when the advancement member is in a home position, at the proximal end portion of the housing; and
wherein the power circuit is transitioned from the open state to the closed state when the advancement member is moved distally from the home position, such that the light generating element receives power from the power circuit and is caused to emit light.

19. The catheter system of claim 14, wherein the light generating element comprises a luminescent coating applied on at least a portion of the instrument, the luminescent coating generating light after exposure to ambient light.

20. The catheter system of claim 14, wherein the housing is formed of a fluorescent plastic.
